(19) United States
(12) Patent Application Publication
(10) Pub. No.: US 2004/0228825 A1
(43) Pub. Date: Nov. 18, 2004

(54) Production method of N-acylamino acid and a salt thereof

(75) Inventors: Yasunori Atarashi, Yokkaichi-shi (JP); Hideki Yoshihara, Yokkaichi-shi (JP); Tatsuya Hattori, Kawasaki-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: AJINOMOTO CO., INC., Tokyo (JP)

(21) Appl. No.: 10/774,073

(22) Filed: Feb. 6, 2004

(30) Foreign Application Priority Data
Feb. 7, 2003 (JP) 31022/2003

(51) Int. Cl. A61K 7/075; A61K 7/08; C07C 233/45

(52) U.S. Cl. 424/70.22; 554/63; 548/338.1; 548/494; 548/530

(57) ABSTRACT

A desired N-acylamino acid and a salt thereof can be produced efficiently and conveniently, almost without leaving any odor in the object product, by reacting amino acid or a salt thereof with a fatty acid halide under basic conditions in the presence of a phosphorus compound.

### TECHNICAL FIELD OF THE INVENTION

The present invention relates to a method for producing N-acylamino acid useful for the production of detergents and the like and a salt thereof.

### BACKGROUND OF THE INVENTION

Amine salt and alkali metal salt of N-long chain acylamino acid have superior surface-activating action and are low irritant. Therefore, they are useful for the production of detergents having a mild action on the skin. As a method for producing N-long chain acylamino acid, a method comprising condensing an amino acid or a salt thereof with a fatty acid halide by the Shotten-Baumann reaction under basic conditions, and then isolating said acylamino acid chain in a free form using an acid is generally known.

In addition, methods for producing N-acyl acidic amino acid from acidic amino acid using a mixed solvent of an organic solvent and water as a reaction solvent are described in JP-B-46-8685 and JP-B-57-47902. To be specific, an acidic amino acid is condensed with a fatty acid halide in a mixed solvent of acetone/water in the presence of alkali, and acyl acidic amino acid is separated by neutral crystallization or layer separation by heating. However, when a product is made using the acyl acidic amino acid obtained by this method, acetone itself, which is a reaction solvent, and odor substances derived from acetone remain in the product, which in turn renders the product not entirely satisfactory in terms of odor. Moreover, such odor cannot be removed completely even if the product is washed.

JP-A-7-157795 and JP-A-5-70418 disclose production methods of acyl amino acid using only an aqueous solvent and free of organic solvent which is one of the causes of odor. However, these methods are not able to produce a high purity product, because the purity of the acylated products obtained by these methods is a little over 90% at maximum. What is more, the viscosity becomes very high in the reaction system used for these methods, and since stirring becomes insufficient at high concentrations, the purity becomes still lower. Therefore, the reaction needs to be carried out at a low concentration where stirring is possible, which means that an object product cannot be provided in large quantity at a time. In addition, the reaction requires a considerable amount of solvent. To conclude, this method involves an economically disadvantageous reaction. As for a method affording an economical reaction at high concentration, JP-A-5-70418 describes a method comprising elevating the reaction temperature along with the progress of acylation. However, this method is also problematic in that the reaction viscosity is high, causing a burden on the facility, and when the stirring is not sufficiently uniform, the purity becomes lower, thus resulting in a high content of fatty acid in N-acylamino acid or a salt thereof obtained by purification, and an odor is ultimately produced.

For removing odor substances, JP-A-3-284658 and JP-A-7-2747 teach a method comprising purification by membrane separation. Nevertheless, this method is not satisfactory because membrane separation is costly, which is economically disadvantageous, and hydrophilic organic solvents causing the odor cannot be removed easily.

It is therefore an object of the present invention to provide a method for efficiently and conveniently producing a desired N-acylamino acid and a salt thereof at high purity, leaving almost no odor in the object product.

### SUMMARY OF THE INVENTION

The present inventors have conducted intensive studies to solve the aforementioned problems and found that, when an amino acid or a salt thereof is reacted with a fatty acid halide to give N-acylamino acid, a reaction under basic conditions in the presence of a phosphorus compound produces N-acylamino acid and a salt thereof almost free of an odor. In addition, this method is efficient and convenient, and the obtained N-acylamino acid and a salt thereof have a high purity.

The present inventors have also found that N-acylamino acid and a salt thereof produced according to the method of the present invention by the use of, for example, acetone, which is considered to cause an odor, as a reaction solvent have almost no odor.

Accordingly, the present invention relates to the following.

[1] A production method of N-acylamino acid or a salt thereof, which comprises a step of reacting an amino acid or a salt thereof with a fatty acid halide under basic conditions in the presence of a phosphorus compound.

[2] The production method of the above-mentioned [1], wherein the phosphorus compound is a reducing phosphorus compound.

[3] The production method of the above-mentioned [2], wherein the reducing phosphorus compound is selected from the group consisting of a phosphorous acid, a hypophosphorous acid and metal salts thereof.

[4] The production method of the above-mentioned [1], wherein the amount of the phosphorus compound to be added is 0.5-38 wt % of the amino acid or a salt thereof.

[5] The production method of the above-mentioned [1], wherein the amino acid is an acidic amino acid.

[6] The production method of the above-mentioned [1], wherein the amino acid is glutamic acid or a sodium salt thereof.

[7] A fragrance cosmetic toiletry product comprising N-acylamino acid or a salt thereof produced by the production method of [1].

[8] A fragrance cosmetic toiletry product comprising N-acylamino acid or a salt thereof produced by the production method of [2].

[9] A fragrance cosmetic toiletry product comprising N-acylamino acid or a salt thereof produced by the production method of [3].

[10] A fragrance cosmetic toiletry product comprising N-acylamino acid or a salt thereof produced by the production method of [4].

### EMBODIMENT OF THE INVENTION

The method of the present invention relates to a method for producing N-acylamino acid and a salt thereof, which comprises a step of reacting an amino acid or a salt thereof with a fatty acid halide, wherein the reaction is carried out under basic conditions in the presence of a phosphorus compound.

The kind of amino acid used in the present invention is not particularly limited and is exemplified by α-amino acid, β-amino acid and the like, which may be acidic, neutral or basic. Concretely, the acidic amino acid is exemplified by glutamic acid, aspartic acid, α-aminoadipic acid, cysteic acid, homocysteic acid and the like; neutral amino acid is exemplified by alanine, cystine, glutamine, glycine, leucine, isoleucine, methionine, phenylalanine, serine, threonine, tryptophan, tyrosine, valine and the like; and basic amino acid is exemplified by arginine, lysine and the like. As the amino acid, a mixture of one or more kinds thereof may be used. As the amino acid, acidic amino acid is preferable, and glutamic acid is particularly preferable. The amino acid to be used in the present invention encompasses amino acid in an optically pure form, any mixture of optical isomers, a racemate, a diastereomer in an optically pure form, any mixture thereof, a mixture of the salts thereof and the like. The salt of amino acid is exemplified by salts with sodium, hydrochloric acid, acetic acid and the like, and glutamic acid sodium salt is particularly preferable. In the following, by the “amino acid” is meant an amino acid or a salt thereof, unless particularly indicated.

The fatty acid halide to be used in the present invention is a saturated or unsaturated fatty acid halide, preferably having 8 to 22, more preferably 8 to 18, carbon atoms. The carbon chain of the fatty acid halide may be linear or branched chain or cyclic, or they may be combined. The carbon chain may have one or more unsaturated bonds. The halogen atom constituting the fatty acid halide may be a chlorine atom, a bromine atom, an iodine atom or a fluorine atom, with preference given to a chlorine atom. A preferable fatty acid halide is that having a linear or branched carbon chain optionally having about one or two double bonds. Concretely, caprylic acid halide, lauric acid halide, myristic acid halide, palmitic acid halide, stearic acid halide, oleic acid halide and the like can be mentioned. In the present invention, not only one kind thereof but a mixture of two or more kinds thereof may be used as a raw material. For example, a mixed fatty acid halide such as coconut oil fatty acid halide, tallowate halide and the like can be used. The total amount of the fatty acid halide to be used is generally preferably 0.5-2 mol, more preferably 0.7-1.1 mol, per. 1 mol of amino acid.

The reaction of an amino acid with a fatty acid halide is carried out in water or in a mixed solvent of a hydrophilic organic solvent and water depending on the kind of the amino acid. Concrete examples of the hydrophilic organic solvent include various alcohols such as ethanol, isopropyl alcohol, 3-butanol, propylene glycol and the like, acetone, triethanolamine and the like. The amount of the solvent to be used and the composition ratio of the mixed solvent are appropriately determined according to the kind of amino acid. For example, when the amino acid is glutamic acid or a salt thereof, the reaction is carried out in a mixed solvent of a hydrophilic organic solvent and water. As used herein, the hydrophilic organic solvent is exemplified by various alcohols such as isopropyl alcohol, 3-butanol, propylene glycol and the like and acetone. The total amount of the reaction solvent to be used for the reaction of an amino acid with a fatty acid halide is not particularly limited as long as the fluidity of the reaction mixture can be maintained. For example, it is generally 5-70 wt %, preferably 15-60 wt %, relative to the total weight of the amino acid and fatty acid halide. While the content of the hydrophilic organic solvent in the reaction solvent is not particularly limited, when, for example, amino acid is glutamic acid or a salt thereof, the concentration of the organic solvent in a mixed solvent of hydrophilic organic solvent/water is preferably 5-60 wt %, more preferably 30-60 wt %, in the stage where amino acid is dissolved before acylation reaction. When the concentration is less than 5 wt %, the acylation ratio becomes low and when it exceeds 60 wt %, the yield of the acylation reaction is fine but recovery of the solvent used is substantially difficult, which in turn defectively increases the product cost.

The reaction of an amino acid with a fatty acid halide is carried out under basic conditions (pH of the reaction mixture preferably being 9-14, more preferably 10-13). The base used to achieve the basic conditions is not particularly limited as to its kind and, for example, metal hydroxide such as sodium hydroxide, potassium hydroxide and the like can be used. The method for adding a base in the present invention includes collective addition and divisional addition. The collective addition means addition at once of an amount to make the pH after completion of the reaction not less than 10, and the divisional addition means addition of divided portions that makes it possible to maintain pH of the reaction mixture at preferably 9-14, more preferably 10-13, during the reaction. When the pH is lower than 9 or higher than 14 during the divisional addition, acylation ratio becomes low, as a result of which the free fatty acid content of the resulting product becomes higher, thereby degrading the quality of the desired acylamino acid. The base may be added in the form of an aqueous solution, where the amount of water then is included in the amount of the reaction solvent to be used for the reaction between the above-mentioned amino acid and fatty acid halide.

The phosphorus compound to be used in the present invention may be inorganic or organic, or reducing or non-reducing, and a reducing phosphorus compound is preferable. The reducing phosphorus compound is a phosphorus compound having an oxidation number on the phosphorus atom of less than +5, such as phosphorous acid, hypophosphorous acid and metal salts thereof. Specific examples of the metal salt include sodium salt, potassium salt, magnesium salt, calcium salt, barium salt and the like. The phosphorus compound may be used alone or in combination of two or more kinds thereof. When used in combination, they may be mixed in advance of the addition or may be added separately. The phosphorus compound is preferably added in a proportion of 0.5-38 wt %, more preferably 0.5-19 wt %, of the amino acid or a salt thereof to be used for the reaction. When the amount of addition is less than 0.5 wt %, the obtained N-acylamino acid and a salt thereof afford a lower odor improvement effect. When it exceeds 38 wt %, an increased amount of the crystal washing water for recovering N-acylamino acid is necessary, thereby possibly exerting an adverse influence on the productivity. When a metal salt is used as a phosphorus compound, the amount of the base to be used for the reaction between amino acid and fatty acid halide can be reduced, which is preferable from the aspect of cost.

The order of addition of the reaction reagents for the reaction of amino acid with fatty acid halide is not particularly limited and may be, for example, mixing amino acid or a salt thereof, a reaction solvent and all amounts of bases necessary for the reaction, and then adding fatty acid chloride to the mixture; or mixing amino acid or a salt thereof, a reaction solvent and a part of the base, and then adding fatty acid chloride and the remaining base such that pH of the reaction mixture is 9-14 during the reaction, or a different order. In the two methods mentioned above, the reaction temperature is −10° C. 70° C., preferably 0° C. to 50° C.

The method for isolating and purifying N-acylamino acid produced in the aforementioned step is not particularly limited, and it can be separated by, for example, adjusting the pH of the reaction mixture after the completion of the reaction to 1-3, and subjecting the precipitated crystals to an existing method (e.g., method using centrifugal separator, pressure and reduced pressure filtration equipment and the like). Washing of the separated crystals removes impurities such as odor components, salts and the like, whereby an object product with a higher purity can be obtained. The solvent to be used for washing is not particularly limited, but when an organic solvent is used, complete removal of the solvent from the product is extremely difficult. Therefore, water is preferably used.

The pH of a reaction mixture can be adjusted using, for example, an acid, and the kind of the acid to be used is not particularly limited. For example, inorganic acids, preferably hydrochloric acid, sulfuric acid and the like, can be used.

The obtained crystals of acylamino acid can be converted to a salt by a conventional method. The salt of acylamino acid is exemplified by alkali metal salts such as sodium, potassium and the like; alkaline earth metal salts such as calcium, magnesium and the like; aluminum salt; zinc salt; ammonium salt; organic amine salts such as monoethanolamine, diethanolamine, triethanolamine and the like; basic amino acid salts such as arginine, lysine and the like; and the like.

The salt of acylamino acid is useful as a surfactant, which can be prepared by, for example, adding an alkaline substance to a free acylamino acid to allow dissolution. The alkaline substance to be used can be appropriately selected from those typically used as surfactants, such as alkali metals (e.g., sodium, potassium etc.), hydroxides thereof, alkaline earth metals (e.g., calcium, magnesium etc.), hydroxides thereof, alkaline substances containing a metal (e.g., aluminum, zinc etc.), hydroxides thereof, organic amines (e.g., ammonia, monoethanolamine, diethanolamine, triethanolamine etc.), basic amino acids (e.g., arginine, lysine etc.), and the like.

A typical use of N-long chain acylamino acid and a salt thereof obtained in the present invention is exemplified by, but not limited to, a material for industrial detergents and processing agents, a material for household (clothes, kitchen, house) detergents, a material for fragrance cosmetic toiletry products and the like.

The fragrance cosmetic toiletry product in the present invention is a collective term of quasi-drugs and cosmetics as defined in the Pharmaceutical Affairs Law, which specifically includes quasi-drugs such as mouth refrigerant, underarm deodorant, bath dusting powders, baldness remedy, hair remover, hair dye, permanent wave agent, bath agent, medicated cosmetics, therapeutic dentifrices and the like; and cosmetics comprising wash cosmetics such as toilet soap, face wash (cream or paste, liquid or gel, granule or powder, aerosol etc.), shampoo, hair rinse and the like, hair cosmetics such as hair color, hair treatment agent (cream, mist, oil, gel and other forms, including split hair coating agent), hair set agent (hair oil, setting lotion, curler lotion, pomade, stick pomade, bintsuke-abura, hair spray, hair mist, hair liquid, hair foam, hair gel, water grease) and the like, basic skin care products such as general cream and milky lotion (cleansing cream, cold cream, vanishing cream, hand cream etc.), shaving cream (after shaving cream, shaving cream etc.), toilet water (hand lotion, general lotion etc.), eau de cologne, shaving lotion (after shaving lotion, shaving lotion etc.), cosmetic oil, face pack and the like, make-up cosmetics such as make-up powder (creamy face powder, pressed face powder, loose face powder, talcum powder, face powder paste, baby powder, body powder, mizu-oshiroi etc.), powder, foundation (cream, liquid, solid etc.), cheek color, eyebrow color, eye cream, eye shadow, mascara and the like, perfumes such as general perfume, perfumed paste, perfumed powder and the like, suntan/sunscreen cosmetics such as suntan/sunscreen cream, suntan/sunscreen lotion, suntan/sunscreen oil and the like, nail cosmetics such as nail cream, enamel, enamel remover and the like, lip cosmetics such as eyeliner cosmetics, lip stick, lip cream and the like, oral cosmetics such as dentifrice and the like, bath cosmetics such as bath salts, bath oil and the like, and the like. Of these, the product of the present invention is often used for the above-mentioned wash cosmetics, hair cosmetics and basic skin care products, particularly preferably wash cosmetics.

In addition, the product of the present invention can be used in combination with various materials used for general fragrance cosmetic toiletry products.

### EXAMPLES

The analysis means and the like used in Example and the like of the present invention are as follows.

(i) Analysis of Acylation Purity of Acylamino Acid Salt-Containing Solution

An acylamino acid salt was quantitatively analyzed by high performance liquid chromatography (HPLC) using a UV detector at a wavelength of 210 nm. The HPLC conditions were as follows:

ODS column (150×60 mm inner diameter), methanol/pH 3.0 aqueous phosphoric acid solution=75/25 (volume ratio) as eluent, eluent flow rate 1.0 mL/min, column temperature 40° C.

The acylation purity can be determined by the following formula.

$$Acylation purity = \frac{\\ Acylamino acid salt weight}{Acylamino acid salt weight + Fatty acid weight} \times 100 ( \% )$$

(ii) Odor Sensory Test

An odor sensory test was based on evaluation of the odor. To be specific, an acylamino acid salt-containing solution (ca. 30 mL) was placed in hard glass bottles with a screw cap (100 mL, diameter 40 mm×height 120 mm), sealed, stood for one day, and smelled by inhouse sensory evaluation expert panelists immediately after opening the cap. The evaluation results of Examples are expressed using ◯ when almost no odor was smelled, Δ when an odor was smelled somewhat, and x when an odor was smelled strongly.

The present invention is explained in detail in the following by referring to Examples, which are not to be construed as limitative.

### Example 1

### Production of an Aqueous N-Coconut Oil Fatty Acid Acylglutamic Acid Triethanolamine Salt-Containing Solution (Sample A):

Sodium glutamate (79 g) was dissolved in an ion-exchange water (99 g) and a 25% aqueous sodium hydroxide solution (55 g), and phosphorous acid (3 g) was added and allowed to dissolve. Acetone (84 g) was added and the liquid temperature was lowered to 10° C. with stirring. A 25% aqueous sodium hydroxide solution (55 g) was added to adjust the solution to pH 11, and coconut oil fatty acid chloride (90 g, 0.40 mol, manufactured by NOF Corporation) was added to this aqueous solution over about 3 hr, while adjusting the solution to pH 10.5-11.5 with a 25% aqueous sodium hydroxide solution (98 g). The reaction temperature then was maintained at 8-15° C. After an acid chloride was added, the mixture was stirred at the same temperature for about 1 hr. The acylation purity of the obtained reaction mixture (508 g) was 95%.

An ion-exchange water (16 g) was added to this reaction mixture (450 g) and 60% sulfuric acid (80 g) was added to adjust its pH to 2, and the mixture was heated to 45° C. Then an ion-exchange water (437 g) was added to this solution and the liquid temperature was lowered to 10° C. to allow precipitation of the crystals. The solution containing the crystals was separated in a compact tabletop centrifugal separator and the crystals were collected and washed with an ion-exchange water (4.0 L). The amount of attached water in the obtained crystals (184 g) was 36%. An ion-exchange water (159 g) was added to the crystals (92 g), heated to 69° C. and neutralized with 90% triethanolamine (30 g) at the same temperature to adjust to pH 5.4 (20° C.). Then, the mixture was adjusted to a concentration of 30% with an ion-exchange water. An ion-exchange water (37 g) was added thereto and the mixture was concentrated under reduced pressure at a liquid temperature of 65° C., which gave an aqueous N-coconut oil fatty acid acylglutamic acid triethanolamine salt-containing solution (sample A, 264 g).

### Example 2

### Production of an Aqueous N-Coconut Oil Fatty Acid Acylglutamic Acid Triethanolamine Salt-Containing Solution (Sample B):

Sodium glutamate (79 g) was dissolved in an ion-exchange water (99 g) and a 25% aqueous sodium hydroxide solution (55 g), and phosphorous acid (0.7 g) was added and allowed to dissolve. Acetone (84 g) was added and the liquid temperature was lowered to 10° C. with stirring. A 25% aqueous sodium hydroxide solution (91 g) was added to adjust the solution to pH 11, and coconut oil fatty acid chloride (90 g, 0.40 mol, manufactured by NOF Corporation) was added to this aqueous solution over about 3 hr, while adjusting the solution to pH 10.5-11.5 with a 25% aqueous sodium hydroxide solution (98 g). The reaction temperature then was maintained at 8-15° C. After an acid chloride was added, the mixture was stirred at the same temperature for about 1 hr. The acylation purity of the obtained reaction mixture (499 g) was 96%.

An ion-exchange water (8 g) was added to this reaction mixture (220 g) and 60% sulfuric acid (38 g) was added to adjust its pH to 2, and the mixture was heated to 45° C. Then an ion-exchange water (214 g) was added to this solution and the liquid temperature was lowered to 10° C. to allow precipitation of the crystals. The solution containing the crystals was separated in a compact tabletop centrifugal separator and the crystals were collected and washed with an ion-exchange water (1.7 L). The amount of attached water in the obtained crystals (92 g) was 40%. An ion-exchange water (135 g) was added to the crystals (91 g), heated to 69° C. and neutralized with 90% triethanolamine (30 g) at the same temperature to adjust to pH 5.3 (20° C.). Then, the mixture was adjusted to a concentration of 30% with an ion-exchange water. An ion-exchange water (37 g) was added thereto and the mixture was concentrated under reduced pressure at a liquid temperature of 65° C., which gave an aqueous N-coconut oil fatty acid acylglutamic acid triethanolamine salt-containing solution (sample B, 234 g)

### Example 3

### Production of an Aqueous N-Coconut Oil Fatty Acid Acylglutamic Acid Triethanolamine Salt-Containing Solution (Sample C):

Sodium glutamate (79 g) was dissolved in an ion-exchange water (99 g) and a 25% aqueous sodium hydroxide solution (55 g), and phosphorous acid (3 g) was added and allowed to dissolve. Acetone (84 g) was added and the liquid temperature was lowered to 10° C. with stirring. A 25% aqueous sodium hydroxide solution (55 g) was added to adjust the solution to pH 11, and coconut oil fatty acid chloride (90 g, 0.40 mol, manufactured by NOF Corporation) was added to this aqueous solution over about 3 hr, while adjusting the solution to pH 10.5-11.5 with a 25% aqueous sodium hydroxide solution (94 g). The reaction temperature then was maintained at 8-15° C. After an acid chloride was added, the mixture was stirred at the same temperature for about 1 hr. The acylation purity of the obtained reaction mixture (503 g) was 95%.

An ion-exchange water (8 g) was added to this reaction mixture (220 g) and 60% sulfuric acid (40 g) was added to adjust its pH to 2, and the mixture was heated to 45° C. Then an ion-exchange water (213 g) was added to this solution and the liquid temperature was lowered to 10° C. to allow precipitation of the crystals. The solution containing the crystals was separated in a compact tabletop centrifugal separator and the crystals were collected and washed with an ion-exchange water (1.7 L). The amount of attached water in the obtained crystals (90 g) was 37%. An ion-exchange water (137 g) was added to the crystals (89 g), heated to 69° C. and neutralized with 90% triethanolamine (30 g) at the same temperature to adjust to pH 5.3 (20° C.). Then, the mixture was adjusted to a concentration of 30% with an ion-exchange water. An ion-exchange water (45 g) was added thereto and the mixture was concentrated under reduced pressure at a liquid temperature of 65° C. This operation was repeated 3 times, which gave an aqueous N-coconut oil fatty acid acylglutamic acid triethanolamine salt-containing solution (sample C, 254 g).

### Example 4

### Production of an Aqueous N-Coconut Oil Fatty Acid Acylglutamic Acid Triethanolamine Salt-Containing Solution (Sample D):

Sodium glutamate (79 g) was dissolved in an ion-exchange water (99 g) and a 25% aqueous sodium hydroxide solution (55 g), and phosphorous acid (14 g) was added and allowed to dissolve. Acetone (84 g) was added and the liquid temperature was lowered to 10° C. with stirring. A 25% aqueous sodium hydroxide solution (55 g) was added to adjust the solution to pH 11, and coconut oil fatty acid chloride (90 g, 0.40 mol, manufactured by NOF Corporation) was added to this aqueous solution over about 3 hr, while adjusting the solution to pH 10.5-11.5 with a 25% aqueous sodium hydroxide solution (146 g). The reaction temperature then was maintained at 8-15° C. After an acid chloride was added, the mixture was stirred at the same temperature for about 1 hr. The acylation purity of the obtained reaction mixture (567 g) was 96%.

An ion-exchange water (16 g) was added to this reaction mixture (450 g) and 60% sulfuric acid (88 g) was added to adjust its pH to 2, and the mixture was heated to 45° C. Then an ion-exchange water (436 g) was added to this solution and the liquid temperature was lowered to 10° C. to allow precipitation of the crystals. The solution containing the crystals was separated in a compact tabletop centrifugal separator and the crystals were collected and washed with an ion-exchange water (3.5 L). The amount of attached water in the obtained crystals (171 g) was 41%. An ion-exchange water (94 g) was added to the crystals (95 g), heated to 69° C. and neutralized with 90% triethanolamine (29 g) at the same temperature to adjust to pH 5.2 (20° C.). Then, the mixture was adjusted to a concentration of 30% with an ion-exchange water. An ion-exchange water (52 g) was added thereto and the mixture was concentrated under reduced pressure at a liquid temperature of 65° C., which gave an aqueous N-coconut oil fatty acid acylglutamic acid triethanolamine salt-containing solution (sample D, 206 g).

### Example 5

### Production of an Aqueous N-Coconut Oil Fatty Acid Acylglutamic Acid Triethanolamine Salt-Containing Solution (Sample E):

Sodium glutamate (79 g) was dissolved in an ion-exchange water (99 g) and a 25% aqueous sodium hydroxide solution (55 g), and 50% aqueous hypophosphorous acid solution (6 g) was added and allowed to dissolve. Acetone (84 g) was added and the liquid temperature was lowered to 10° C. with stirring. A 25% aqueous sodium hydroxide solution (55 g) was added to adjust the solution to pH 11, and coconut oil fatty acid chloride (90 g, 0.40 mol, manufactured by NOF Corporation) was added to this aqueous solution over about 3 hr, while adjusting the solution to pH 10.5-11.5 with a 25% aqueous sodium hydroxide solution (101 g). The reaction temperature then was maintained at 8-15° C. After an acid chloride was added, the mixture was stirred at the same temperature for about 1 hr. The acylation purity of the obtained reaction mixture (514 g) was 96%.

An ion-exchange water (16 g) was added to this reaction mixture (450 g) and 60% sulfuric acid (81 g) was added to adjust its pH to 2, and the mixture was heated to 45° C. Then an ion-exchange water (437 g) was added to this solution and the liquid temperature was lowered to 10° C. to allow precipitation of the crystals. The solution containing the crystals was separated in a compact tabletop centrifugal separator and the crystals were collected and washed with an ion-exchange water (3.5 L). The amount of attached water in the obtained crystals (193 g) was 42%. An ion-exchange water (93 g) was added to the crystals (95 g), heated to 69° C. and neutralized with 90% triethanolamine (29 g) at the same temperature to adjust to pH 5.2 (20° C.). Then, the mixture was adjusted to a concentration of 30% with an ion-exchange water. An ion-exchange water (53 g) was added thereto and the mixture was concentrated under reduced pressure at a liquid temperature of 65° C., which gave an aqueous N-coconut oil fatty acid acylglutamic acid triethanolamine salt-containing solution (sample E, 203 g).

### Example 6

### Production of an Aqueous N-Coconut Oil Fatty Acid Acylglutamic Acid Triethanolamine Salt-Containing Solution (Sample F):

Sodium glutamate (79 g) was dissolved in an ion-exchange water (99 g) and a 25% aqueous sodium hydroxide solution (55 g), and hypophosphorous acid (3 g) was added and allowed to dissolve. Acetone (84 g) was added and the liquid temperature was lowered to 10° C. with stirring. A 25% aqueous sodium hydroxide solution (55 g) was added to adjust the solution to pH 11, and coconut oil fatty acid chloride (90 g, 0.40 mol, manufactured by NOF Corporation) was added to this aqueous solution over about 3 hr, while adjusting the solution to pH 10.5-11.5 with a 25% aqueous sodium hydroxide solution (93 g). The reaction temperature then was maintained at 8-15° C. After an acid chloride was added, the mixture was stirred at the same temperature for about 1 hr. The acylation purity of the obtained reaction mixture (503 g) was 96%.

An ion-exchange water (16 g) was added to this reaction mixture (450 g) and 60% sulfuric acid (81 g) was added to adjust its pH to 2, and the mixture was heated to 45° C. Then an ion-exchange water (437 g) was added to this solution and the liquid temperature was lowered to 10° C. to allow precipitation of the crystals. The solution containing the crystals was separated in a compact tabletop centrifugal separator and the crystals were collected and washed with an ion-exchange water (3.5 L). The amount of attached water in the obtained crystals (95 g) was 42%. An ion-exchange water (92 g) was added to the crystals (92 g), heated to 69° C. and neutralized with 90% triethanolamine (27 g) at the same temperature to adjust to pH 5.2 (20° C.). Then, the mixture was adjusted to a concentration of 30% with an ion-exchange water. An ion-exchange water (53 g) was added thereto and the mixture was concentrated under reduced pressure at a liquid temperature of 65° C., which gave an aqueous N-coconut oil fatty acid acylglutamic acid triethanolamine salt-containing solution (sample F, 202 g).

### COMPARATIVE EXAMPLE 1

### Production of an Aqueous N-Coconut Oil Fatty Acid Acylglutamic Acid Triethanolamine Salt-Containing Solution (Sample G) (Free of Phosphorus Compound):

Sodium glutamate (79 g) was dissolved in an ion-exchange water (99 g) and a 25% aqueous sodium hydroxide solution (55 g). Acetone (84 g) was added and the liquid temperature was lowered to 10° C. with stirring. A 25% aqueous sodium hydroxide solution (55 g) was added to adjust the solution to pH 11, and coconut oil fatty acid chloride (90 g, 0.40 mol, manufactured by NOF Corporation) was added to this aqueous solution over about 3 hr, while adjusting the solution to pH 10.5-11.5 with a 25% aqueous sodium hydroxide solution (91 g). The reaction temperature then was maintained at 8-15° C. After an acid chloride was added, the mixture was stirred at the same temperature for about 1 hr. The acylation purity of the obtained reaction mixture (498 g) was 95%.

An ion-exchange water (8 g) was added to this reaction mixture (220 g) and 60% sulfuric acid (37 g) was added to adjust its pH to 2, and the mixture was heated to 45° C. Then an ion-exchange water (214 g) was added to this solution and the liquid temperature was lowered to 10° C. to allow precipitation of the crystals. The solution containing the crystals was separated in a compact tabletop centrifugal separator and the crystals were collected and washed with an ion-exchange water (1.7 L). The amount of attached water in the obtained crystals (92 g) was 40%. An ion-exchange water (129 g) was added to the crystals (91 g) heated to 69° C. and neutralized with 90% triethanolamine (29 g) at the same temperature to adjust to pH 5.3 (20° C.). Then, the mixture was adjusted to a concentration of 30% with an ion-exchange water. An ion-exchange water (35 g) was added thereto and the mixture was concentrated under reduced pressure at a liquid temperature of 65° C., which gave an aqueous N-coconut oil fatty acid acylglutamic acid triethanolamine salt-containing solution (sample G, 235 g).

The aqueous N-coconut oil fatty acid acylglutamic acid triethanolamine salt-containing solution obtained in Examples 1-6 and Comparative Example 1 were subjected to the measurement of acylation purity and an odor test (sensory test). These results are shown in Table 1. As is clear from the odor test in Table 1, comparison of addition of a phosphorus compound (Examples 1-6) and non-addition (Comparative Example 1) reveals that the obtained aqueous N-coconut oil fatty acid acylglutamic acid triethanolamine salt-containing solutions were almost free of an odor.

TABLE 1

| | Aqueous N-coconut oil fatty acid | | |
|---|---|---|---|
| | acylglutamic acid triethanolamine salt- | | |
| | containing solution | | |
| | amount of | | |
| | phosphorus | | odor test |
| | compound added | acylation | (sensory |
| | (wt %) * | purity (%) | test) |
| Example 1 (sample A) | 3.8 | 95 | ∘ |
| Example 2 (sample B) | 0.9 | 97 | ∘ |
| Example 3 (sample C) | 3.8 | 94 | ∘ |
| Example 4 (sample D) | 17.7 | 96 | ∘ |
| Example 5 (sample E) | 3.8 | 96 | ∘ |
| Example 6 (sample F) | 3.8 | 96 | ∘ |
| Comparative Example 1 | 0.0 | 95 | x |
| (sample G) | | | |

### FORMULATION EXAMPLE

### Hair Shampoo

| | (wt %) |
|---|---|
| Cocoyl glutamic acid triethanolamine salt solution | 35.0 |
| (sample C) | |
| Coconut fatty acid diethanolamide*1 | 5.0 |
| PCA soda (50%) | 0.3 |
| Chlorinated O-[2-hydroxy-3- | |
| (trimethylammonio)propyl]hydroxyethyl cellulose *2 | 0.3 |
| 1,3-butylene glycol | 3.0 |
| Methyl p-oxybenzoate | 0.2 |
| Salicylic acid | 0.2 |
| Stearyl hydroxymyristylene ether*3 | 2.7 |
| Purified water | rest |
| total | 100.0 |

*1: Amisol CDE (manufactured by Kawaken Fine Chemicals Co., Ltd.)
*2: Leoguard GP (manufactured by Lion Corporation)
*3: Elfacos GT-282S (manufactured by Lion Corporation)

The above-mentioned components were heated at 65° C. and dissolved to give a shampoo. This shampoo showed sufficient washing performance and comfortability during use with only a small odor.

### INDUSTRIAL APPLICABILITY

According to the production method of the present invention, the odor of N-acylamino acid and a salt thereof obtained by reacting amino acid or a salt thereof with a fatty acid halide can be drastically reduced. In addition, the production method of the present invention is efficient and convenient.

This application is based on a patent application No. 31022/2003 filed in Japan, the contents of which are hereby incorporated by reference.

What is claimed is:

1. A production method of N-acylamino acid or a salt thereof, which comprises a step of reacting an amino acid or a salt thereof with a fatty acid halide under basic conditions in the presence of a phosphorus compound.

2. The production method of claim 1, wherein the phosphorus compound is a reducing phosphorus compound.

3. The production method of claim 2, wherein the reducing phosphorus compound is selected from the group consisting of a phosphorous acid, a hypophosphorous acid and metal salts thereof.

4. The production method of claim 1, wherein the amount of the phosphorus compound to be added is 0.5-38 wt % of the amino acid or a salt thereof.

5. The production method of claim 1, wherein the amino acid is an acidic amino acid.

6. The production method of claim 1, wherein the amino acid is glutamic acid or a sodium salt thereof.

7. A fragrance cosmetic toiletry product comprising N-acylamino acid or a salt thereof produced by the production method of claim 1.

8. A fragrance cosmetic toiletry product comprising N-acylamino acid or a salt thereof produced by the production method of claim 2.

9. A fragrance cosmetic toiletry product comprising N-acylamino acid or a salt thereof produced by the production method of claim 3.

10. A fragrance cosmetic toiletry product comprising N-acylamino acid or a salt thereof produced by the production method of claim 4.
